(19) United States
(12) Patent Application Publication
(10) Pub. No.: US 2023/0320398 A1
(43) Pub. Date: Oct. 12, 2023

(54) STABILIZED VITAMIN A AND METHOD OF PRODUCTION

(71) Applicant: PARTICLES FOR HUMANITY, PBC, West Lebanon, NH (US)

(72) Inventors: Donald E. CHICKERING, III, Framingham, MA (US); Samantha W. BRADY, Framingham, MA (US); Julie STRAUB, Winchester, MA (US); Jérôme VALLEJO, Granville (FR); Julie WYNS, Acigné (FR); Elsa Abou JAOUDE, Rennes (FR); Haisong YANG, Wheaton, IL (US)

(73) Assignee: PARTICLES FOR HUMANITY, PBC, West Lebanon, NH (US)

(21) Appl. No.: 18/296,681

(22) Filed: Apr. 6, 2023

Related U.S. Application Data
(60) Provisional application No. 63/328,057, filed on Apr. 6, 2022.

(51) Int. Cl. A23L 33/155; A23L 23/10; A23L 29/10; A23L 29/212

(52) U.S. Cl. CPC A23L 33/155; A23L 23/10; A23L 29/10; A23L 29/212

(57) ABSTRACT

Methods are provided for producing fat-soluble vitamin particles. The particles exhibit extended shelf life and cooking stability and can be made without mineral acids or organic solvents. The particles are composite particles of a pH sensitive polymer and a fat-soluble vitamin such as vitamin A. The particles can be incorporated into foodstuffs such as bouillon, cereals, wheat flour, millet flour, cassava flour, tapioca flour, teff flour, corn meal, milk, milk powder, malt beverages, soy sauce, ready-to-use therapeutic foods, rice, or sugar. The foods can provide stable sources of vitamin A for populations in need thereof.

### TECHNICAL FIELD

This disclosure relates to the production of fat-soluble vitamins and, in particular, to the production of stabilized vitamin A.

### BACKGROUND

Vitamin deficiency continues to be a problem, particularly in lower income countries. Many in these countries have diets that do not provide adequate amounts of vitamin A, and fortification of foodstuffs with vitamin A can be difficult due to poor shelf life and cooking degradation of added vitamin A. There is a need for improved forms of vitamin A that can withstand storage at high temperatures and/or humidity and can maintain bioactivity levels after cooking.

### SUMMARY OF THE INVENTION

In one aspect, a composite particle of vitamin A, or a derivative thereof, is provided, the particle comprising a pH sensitive polymer, and subparticles or subdroplets of vitamin A embedded in the pH sensitive polymer, wherein the composite particle is essentially free of organic solvents and mineral acids and the amount of vitamin A in the composite particle is a first concentration after production of the particle and a second concentration after exposing the composite particle to both a shelf life test at 40° C. for 4 weeks and a cooking test in water at 90° C. for 2 hours, and the second concentration is more than 60%, more than 70%, more than 80% or more than 90% of the first concentration. The composite particle can include vitamin A palmitate, ascorbic acid, maltodextrin, modified starch, ascorbic acid, BHT, BHA, tocopherol, or a combination thereof. It can be essentially free of surfactants. The pH sensitive polymer can be a polymethacrylate such as BMC. The subparticles or subdroplets can have a median volume diameter D50 of between 50 nm and 2 μm, between 100 nm and 1 μm, between 100 nm and 500 nm, or between 200 nm and 400 nm, and the composite microparticles can have a median volume diameter, D50, of between 1 and 1000 μm, 1 and 100 μm, 1 and 10 μm, 50 and 1000 μm, 50 and 400 μm, 100 and 400 μm, 50 and 300 μm or 100 and 500 μm with a standard deviation of less than 50 microns. The composite particle can include vitamin C. The composite particle can be added to bouillon and foodstuffs including wheat flour, millet flour, cassava flour, tapioca flour, teff flour, corn meal, milk, milk powder, malt beverages, soy sauce, ready-to-use therapeutic foods, rice, or sugar.

In another aspect, a method is provided, the method comprising forming an acidified aqueous vehicle at a pH of less than or equal to 6.0, combining a pH sensitive polymer with the aqueous vehicle to form a colloidal suspension, allowing the pH of the suspension to rise to greater than 6.0, emulsifying a fat-soluble vitamin or derivative thereof, combining the colloidal suspension and the emulsified fat-soluble vitamin or derivative thereof, limiting the increase in pH after addition of the fat-soluble vitamin or derivative thereof to a pH of 7.0, and removing water from the dispersion to produce composite microparticles comprising a matrix of fat-soluble vitamin, or derivatives thereof, and a pH sensitive polymer. The water can be removed by spray drying, fluid bed drying, vacuum drying, rotary evaporator drying, lyophilizing and/or multistage drying. The method can be free of surfactants and/or mineral acids. The pH of the acidified aqueous vehicle can be reduced to 5.5 or less using an organic acid and the organic acid can be ascorbic acid, tartaric acid or both. The method can avoid the use of organic solvents. The fat-soluble vitamin can be emulsified in an aqueous solution comprising maltodextrin, starch, or a combination thereof. The method can include coating the composite microparticles with a polysaccharide and the polysaccharide can be starch, modified starch and/or maltodextrin. The particles produced can have a median volume diameter, D50, of greater than 1 μm and less than 500 μm, greater than 50 μm and less than 400 μm, greater than 100 μm and less than 200 μm. The method can include adding the composite microparticles to a food and adding an antioxidant to the fat-soluble vitamin. The antioxidant can be a synthetic antioxidant selected from at least one of BHA and BHT. The method can include adding a defoaming or antifoaming agent.

In another aspect a food ingredient is provided, the food ingredient comprising composite microparticles comprising subparticles or subdroplets of vitamin A or a derivative thereof in a pH sensitive polymer, the composite microparticles having a median volume diameter, D50, of between 1 and 1000 μm, 1 and 100 μm, 1 and 10 μm, 50 and 1000 μm, 50 and 400 μm, 100 and 400 μm, 50 and 300 μm or 100 and 500 microns wherein the composite microparticles are essentially free of anionic surfactants. It can include ascorbic acid, BHT, BHA, tocopherol, or a combination thereof and can be manufactured without the use of a mineral acid. It can be used in bouillon, wheat flour, millet flour, cassava flour, tapioca flour, teff flour, corn meal, milk, milk powder, malt beverages, soy sauce, ready-to-use therapeutic foods, rice, or sugar. It can be essentially free of mineral acids, surfactants and organic solvents. The pH sensitive polymer can be a polymethacrylate such as BMC. It can include vitamin A palmitate, ascorbic acid, maltodextrin and modified starch. The subparticles or subdroplets can exhibit a median volume diameter D50 of between 50 nm and 2 μm, between 100 nm and 1 μm, between 100 nm and 500 nm, or between 200 nm and 400 nm. The composite microparticles can have a standard deviation of less than 50 microns. The food ingredient can include soluble vitamins such as vitamin C.

In another aspect a food ingredient comprises composite microparticles including subparticles or subdroplets of vitamin A or a derivative thereof in a matrix of a pH sensitive polymer, the composite microparticles having a median volume diameter D50 of between 1 and 1000 μm, 1 and 100 μm, 1 and 10 μm, 50 and 1000 μm, 50 and 400 μm, 100 and 400 μm, 50 and 300 μm or 100 and 500 μm wherein the composite microparticles comprise a synthetic antioxidant. The synthetic antioxidant can be BHA and/or BHT. The food ingredient can include vitamin C and can be manufactured without the use of mineral acids or anionic surfactants. It can be essentially free of mineral acids and organic solvents. It can be incorporated into bouillon, wheat flour, millet flour, cassava flour, tapioca flour, teff flour, corn meal, milk, milk powder, malt beverages, soy sauce, ready-to-use therapeutic foods, rice, or sugar. The pH sensitive polymer can be a polymethacrylate such as BMC. It can include vitamin A palmitate, ascorbic acid, maltodextrin and modified starch. The composite microparticles can have a standard deviation of less than 50 μm.

In another aspect a method is provided, the method comprising mixing a pH sensitive polymer with water and an organic acid to produce a suspension, adding at least one polysaccharide to the suspension, adding a fat-soluble vitamin or derivative thereof to the suspension, adjusting the suspension to a pH of less than 6.0, emulsifying the suspension to produce composite droplets having a median volume diameter D50 of less than 1 μm, limiting a rise in pH to less than 8.0, and removing water from the emulsion to produce composite microparticles comprising a matrix of fat soluble vitamin, or derivatives thereof, and a polymethacrylate polymer binder. The method can include allowing the pH of the suspension to rise to a pH of at least 6.0. The fat-soluble vitamin or derivative thereof can be emulsified in an aqueous vehicle comprising one or more polysaccharides and an organic acid. The one or more polysaccharides can be maltodextrin and/or modified starch, and the organic acid can be ascorbic acid and/or tartaric acid. The pH sensitive polymer can be a polymethacrylate such as BMC.

In another aspect, a method is provided, the method including preparing an aqueous suspension of at least one polysaccharide and an organic acid, adding a fat-soluble vitamin or derivative thereof to the suspension, mixing a solid pH sensitive polymer into the suspension, adjusting the emulsion to a pH of less than 6.0, emulsifying the suspension to produce composite droplets having a median volume diameter D50 of less than 1 μm, and removing water from the emulsion to produce composite microparticles comprising a matrix of fat-soluble vitamin, or derivatives thereof, and a polymethacrylate polymer binder. The one or more polysaccharides can be maltodextrin and/or modified starch, and the organic acid can be ascorbic acid and/or tartaric acid. The pH sensitive polymer can be a polymethacrylate such as BMC. Any of the methods can use water having a hardness of less than 100 ppm as CaCO3, such as tap water having a hardness of less than 100 ppm as CaCO3. The compositions and methods described herein may be used separately or together, and components or techniques described in relation to one system or method are capable of being implemented with the others. The subject matter of this application may involve, in some cases, interrelated products, alternative solutions to a particular problem, and/or a plurality of different uses of a single system or article.

### BRIEF DESCRIPTION OF THE DRAWINGS

In the figures,

FIG. 1 is a schematic diagram and flow chart illustrating one embodiment of a system for processing and drying composite vitamin particles;

FIG. 2 is a schematic diagram illustrating a composite particle;

FIG. 3 is a schematic diagram illustrating one embodiment of an emulsification process;

FIG. 4 is a schematic diagram illustrating a second embodiment of an emulsification process;

FIG. 5 is a schematic diagram illustrating a third embodiment of an emulsification process;

FIG. 6 illustrates graphically data comparing the shelf life of comparative VAP samples and a comparative commercial VAP samples;

FIG. 7 illustrates graphically the cooking stability of the VAP samples of FIG. 6;

FIG. 8 illustrates graphically data comparing the shelf life of experimental VAP samples and a comparative commercial VAP sample;

FIG. 9 illustrates graphically the cooking stability of the VAP samples of FIG. 8;

FIG. 10 illustrates graphically data comparing the shelf life of experimental VAP samples and a comparative commercial VAP sample; and

FIG. 11 illustrates graphically the cooking stability of the VAP samples of FIG. 10.

The above-mentioned and other features of this disclosure, and the manner of attaining them, will become more apparent and better understood by reference to the following description of embodiments described herein taken in conjunction with the accompanying drawings, wherein:

### DETAILED DESCRIPTION

Disclosed herein are stabilized particles of vitamin A, foodstuffs fortified with vitamin A, and methods of producing stabilized vitamin A. In a first aspect, methods are disclosed for producing particles of vitamin A and various foods in which the particles can be incorporated. The particles can be mass produced at a consistent size so that they have predictable stability on the shelf, during cooking, and in the body. When incorporated into foodstuffs such as bouillon, cereals, wheat flour, millet flour, cassava flour, tapioca flour, teff flour, corn meal, milk, milk powder, malt beverages, soy sauce, ready-to-use therapeutic foods, rice, or sugar, the vitamin A retains greater activity during storage and in cooking when compared to previously available vitamin A particles. The particles can also be incorporated into animal feed and non-foodstuffs such as supplements, micronutrient powders, therapeutics, skin treatments and cosmetics.

The processes disclosed herein involve forming a stable particle of a fat-soluble vitamin in the absence of a non-aqueous solvent. Furthermore, the pH sensitive polymer used to encapsulate and protect the vitamin A need not be water soluble. A fat-soluble vitamin in an aqueous system is combined with a water insoluble pH sensitive polymer in an aqueous system and is manipulated into a stable, bio-available food additive. In one aspect, vitamin A microdroplets can be produced in an emulsion and dried to form microparticles. As used herein “microdroplets” refer to droplets of vitamin A produced during the emulsion process, and “microparticles” refer to particles produced during the drying process. Particles having a diameter of less than 1 μm are nanoparticles. A “subparticle” is a particle or droplet that is contained in a larger composite particle. Particles can include a polymer that releases vitamin A after it is ingested. The polymer can be natural or synthetic and can be pH sensitive, meaning that it is stable at one pH range and unstable at another. The vitamin A particles can be produced without the use of organic solvents, without the use of mineral oil, without the use of mineral acids and without the use of undesirable surfactants. The absence of these materials provides for a safer product that can be safely incorporated into foodstuffs. The examples provided herein are directed to vitamin A but other nutrients and fat-soluble vitamins may also be processed using the methods described. Other fat-soluble vitamins include vitamins D, E and K.

The production processes can be purely aqueous processes. They can use organic acids to lower pH, and the processes can be void of mineral acids. A process that is void of mineral acids is a process that does not include the addition of a mineral acid at any point in the particle production process. To reduce pH and promote the dissolution or dispersion of polymers and other components, organic acids can be used. In some cases, the organic acid is a vitamin. Suitable acids can have a pKa of greater than 2.5, greater than 2.9, greater than 3.5, greater than 3.75, greater than 4.0, greater than 4.25, greater than 4.5, greater than 4.75, less than 5.5, less than 5, less than 4.5, less than 4.25, less than 4.0, less than 3.75 or less than 3.5. Exemplary organic acids include ascorbic acid, tartaric acid, folic acid, stearic acid, acetylsalicylic acid, citric acid and oleic acid. pH buffers may be included but can also be excluded from the process. The processes described herein can also be carried out without the use of surfactants that may be undesirable in foodstuffs and the processes can be essentially free of these surfactants. These surfactants include sodium dodecyl sulfate (SDS), sodium lauryl sulfate (SLS), stearyl alcohol, polysorbate, polyethylene glycol stearate, cetearyl glucoside, polyglyceryl stearate, polyglyceryl distearate, sodium stearoyl glutamate, distearyldimethyl ammonium chloride. A composition is “essentially free” of a compound if it comprises non-detectable levels of the compound or, if the compound is detected, it is at levels that are attributable only to background levels of the compound in components used to produce the particle. The water used in the processes can be of different purities in different embodiments. For example, the water can be ultrapure, reverse osmosis treated, carbon filtered, distilled or tap water. The water can have a hardness of less than 50, 100 or 200 ppm CaCO₃.

Vitamin A is a fat-soluble vitamin that can be present in three active forms including retinol, retinal and retinoic acid. As used herein, “vitamin A” includes these forms and those compounds that can be converted in vivo to vitamin A. These structures include, for example, carotenoids, retinyl palmitate, retinyl acetate, all-trans-retinol, all-trans-retinal, all-trans-retinoic acid, 11-cis-retinal, 13-cis-retinoic acid and 9-cis-retinoic acid. Carotenoids include α-carotene, β-carotene, β-cryptoxanthin, lutein, zeaxanthin and lycopene.

The process may include the addition of antioxidants to help prolong the activity of the vitamin A. Antioxidants should be selected from those that are safe for human and/or animal consumption. Antioxidants can be added at any time during the production process, for example, in the raw materials, at the emulsion stage, at the particle drying stage, upon bulk packaging, or when combined with foodstuffs. Antioxidants are provided at a concentration that is adequate to protect the vitamin A from decomposition. The ratio of antioxidant to vitamin A on a w/w basis can be, for example, greater than 1:100, greater than 1:20, greater than 1:10, greater than 1:5, less than 1:2, less than 1:4, less than 1:10 or less than 1:20. Antioxidants can be synthetic or natural. Such antioxidants can include, for example, tocopherol, butylated hydroxyanisole (BHA), butylated hydroxytoluene (BHT), tert-butylhydroquinone (TBHQ), anoxomer, dilauryl thiodipropionate, ethoxyquin, nordihydroguaiaretic acid, propyl gallate, octyl gallate, 2,4,5-trihydroxybutyrophenone (THBP), ascorbyl palmitate, sodium ascorbate, calcium ascorbate, ascorbic acid, rosemary extract, and/or thiodipropionic acid.

The processes can be entirely aqueous based and can be void of organic solvents. As used herein, organic solvents are those hydrocarbon-based solvents, both aliphatic and aromatic, having a solubility of less than 10% w/w in water at room temperature. Specific organic solvents among those that can be excluded include acetone, chloroform, methylene chloride, toluene and tetrahydrofuran. In addition to being free of organic solvents, the processes can also avoid the use of alcohols, aldehydes and ketones. In some embodiments, the aqueous process is completed without the use of methylene chloride or acetone, or without both of methylene chloride and acetone. In some embodiments, the materials used can be limited to water, organic acids, pH sensitive polymers, antioxidants and excipients approved for foodstuffs.

Vitamin particles disclosed herein can be associated with a polymeric coating such as a pH sensitive polymer that protects the vitamin A from degradation but allows the vitamin A to become bioavailable in the human gut and/or animal gut. The pH sensitive polymer can be non-toxic and should be suitable for human consumption. As used herein, a polymer is pH sensitive if it is unaffected at a first pH but at a second pH (the pH critical point) degrades, swells, dissolves or otherwise changes form to a degree where a substance surrounded by the polymer is exposed to the environment. Typically, a pH sensitive polymer is subject to degradation, dispersion or dissolution at a lower pH. For example, a polymer that is pH sensitive at a pH of 6.0 would be intact at a pH above 6.0 and would release material at a pH below 6.0. In various embodiments, the polymers used herein can degrade or dissolve at a pH below 6.0, below 5.5, below 5.0, below 4.5, below, 4.0, below 3.5 or below 3.0. In this and other embodiments, the polymer can be stable and insoluble at a pH greater than 3.0, greater than 3.5, greater than 4.0, greater than 4.5, greater than 5.0, greater than 5.5, or greater than 6.0.

The pH sensitive polymers described herein can include basic groups, such as amine groups, or acidic groups, such as carboxylic acid, phosphonic acid and sulfonic acid groups. The polymers can be natural polymers such as cellulosic or polysaccharide polymers. The polymers can include a hydrolytically active polycarbonate backbone. In one set of embodiments, the polymer used includes acidic groups and can be a cationic copolymer such as a polymethacrylate. In specific embodiments the polymer can be an ethyl methacrylate-methacrylic acid copolymer based on dimethylaminoethyl methacrylate, butyl methacrylate and methyl methacrylate at a ratio of, for example, 2:1:1. The pH sensitive polymer can have a molecular weight in the range of less than 50,000 g/mol, less than 100,000 g/mol, from 25,000 to 75,000 g/mol, from 100,000 to 200,000 g/mol, from 200,000 to 300,000 g/mol, from 300,000 to 400,000 g/mol, from 400,000 to 500,000 g/mol or less than 500,000 g/mol. In one embodiment, the pH sensitive polymer is ethyl methacrylate-methacrylic acid copolymer based on dimethylaminoethyl methacrylate, butyl methacrylate and methyl methacrylate at a ratio of 2:1:1 and having an average molecular molar mass of 47,000. These polymers are commonly referred to as basic methacrylate copolymer or BMC. These polymers are available from Evonik under the tradenames EUDRAGIT® and EUDRAGUARD® in powder form. The polymers are available in different particle sizes and in many embodiments the smaller sized particles are preferred. For example, particles that are less than 1 μm or 10 μm in diameter (EUDRAGIT E PO or EUDRAGUARD Protect) have been used to more quickly provide a workable suspension of the material.

The concentration of vitamin A in the dried particles can be greater than 1%, greater than 10%, greater than or equal to 12%, greater than or equal to 14%, greater than or equal to 15%, greater than or equal to 16%, greater than or equal to 18%, greater than or equal to 20%, greater than or equal to 22% or greater than or equal to 24% by mass. In addition, the amount of fat soluble vitamin (vitamin A) in relation to the pH sensitive polymer, by weight, can be greater than or equal to 0.1:1, 1:1, 1.2:1, 1.5:1, 2:1, or 10:1. In addition to vitamin A and the pH sensitive polymer, the balance of the particles (approx. 10-90%) can comprise, for example, starch, modified starch, maltodextrin, sugar, gum arabic, antioxidants, aqueous soluble vitamins such as vitamin C, fat soluble vitamins such as vitamins D and K, and colorants.

Additional materials that can be used in the particles include excipients such as colorants, mixing aids, emulsifiers, antifoaming agents, defoaming agents, and structural enhancers such as starch, modified starch and maltodextrin. In some embodiments, excipients can improve the process and/or product. For instance, a modified starch can improve the suspension of vitamin A in aqueous systems, and in combination with vitamin A (liquid) it can produce an entity that interacts with the pH sensitive polymer to a greater degree than in the absence of the modified starch. Modified starches include materials made from corn, waxy maize, tapioca/cassava, potato, or wheat, and can exhibit a pH of between 3.5 and 4.5 in a 9% aqueous slurry. One of these modified starches is available commercially as HI-CAP® 100. In other embodiments, excipients can improve the process. In some cases, incorporation of a pH sensitive polymer in an aqueous system can result in the generation of foam within mixes, which can decrease emulsion quality, impede subsequent ingredient addition, reduce product yield, and/or produce dried particles of lower quality and/or of greater variability. Addition of antifoaming and/or defoaming agents can prevent formation of foam and/or break up formation of foam within mixtures by reducing the surface tension of droplets at the surface. Antifoaming/defoaming agents can be oil-based, water-based, or silicon-based, and can involve ingredients such as vegetable oil, mono-di-glycerides, polydimethyl siloxane, and silicon dioxide. One of these defoaming agents is available commercially as MAGRABAR® MD-3000.

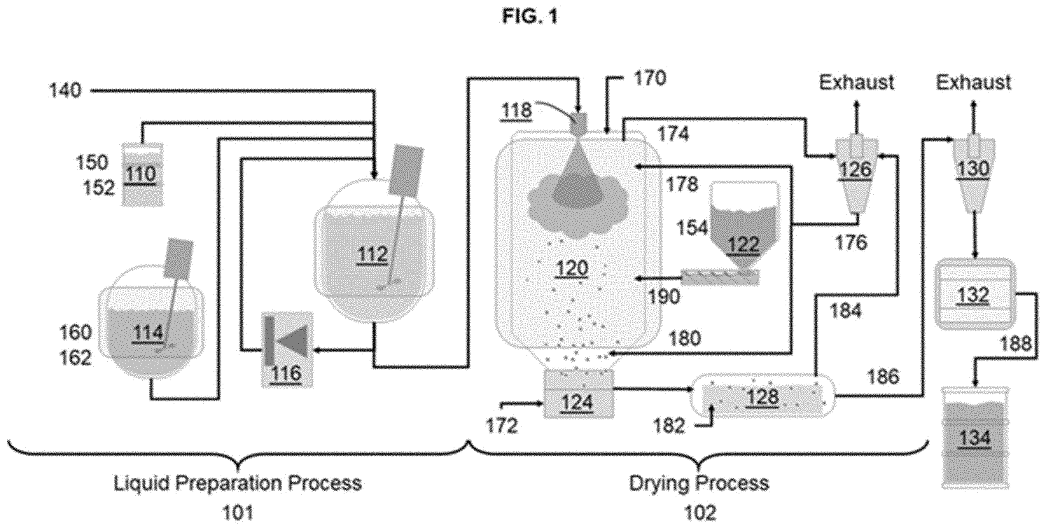

One set of embodiments is illustrated in the diagram of FIG. 1. The equipment and techniques illustrated in FIG. 1 provide an example of how the methods can be employed. As shown, the process is divided into liquid preparation process 101 and drying process 102. All components can be fed to emulsion vat 112 and are passed through high-pressure homogenizer 116. Both the vitamin and the polymer components can be in the form of a liquid at room temperature and can be mixed together. Liquid preparation process 101 uses container 110 into which the vitamin A (retinol palmitate, e.g.) 150 and antioxidant 152 are measured. The polymer can be dispersed in mixer 114 and the organic acid can be added before or after the polymer has been added to the mixer 114. Any excipients, such as a modified starch, can be added via stream 140. The polymer 160, such as EUDRAGUARD Protect, is mixed in powder form as received with a fixed amount of organic acid 162 in water in mixer 114. The amount of water used should be adequate to form a useable suspension of the polymer and can be, for example, between 2 and 20 times the amount of polymer, by weight. The temperature of the water can be between 10-50° C., 20-40° C., 30-40° C., or 30-35° C. The initial pH of the mixture immediately after the mixing of the acid and polymer can be, for example, less than 7.5, less than 7.0, less than 6.5, less than 6.0, less than 5.5, less than 5.0, less than 4.5 or less than 4.0. Specific ranges for initial pH can be 3.0 to 5.0, 3.5 to 5.0, 4.0 to 5.5, 4.0 to 5.0, 4.5 to 5.0, 5.0 to 6.0, and 6.0 to 7.0. The amount of organic acid mixed with the polymer can be provided as a ratio of moles of organic acid to kg of polymer. This ratio can be, in various embodiments, in a range of from 1:10 to 2:1, 1:5 to 5:1, 1:3 to 2:1, 1:2 to 5:1, 1:1 to 1:1.2, or 1.05:1 to 1.20:1. For example, 1.2 mols of ascorbic acid per kg of EUDRAGUARD Protect per 5 L water achieves an initial pH between 4.5 and 5.0. As the solid polymer particles dissociate and the polymer molecules become suspended, the pH of the mixture rises. To improve droplet and particle morphology, the pH is allowed to rise to greater than 5.5, greater than 6.0, greater than 6.5 or greater than 7.0. In some embodiments, the pH rise is not manipulated by adding any base or buffer after the initial mixing but is regulated only by the fixed amount of acid added at the initiation of the mixing process and the rise in pH attributable to the polymer. The mixture is agitated until partial or full clarification of the solution. Clarification may be determined by measuring turbidity and in various embodiments exhibits a threshold of less than 100, less than 50, less than 40, less than 30, less than 20, less than 10, less than 5 or less than 2 nephelometric turbidity units (NTU). This process can take less than 24 hr, less than 12 hr, less than 3 hr, or less than 1 hr. In some embodiments, the suspension is clarified in about 30 minutes. The suspension can be used immediately or can be stored stably for days, weeks or months prior to forming dry particles. A suspension is considered to be stable if it does not separate into distinct phases.

Suspended polymer from mixer 114, vitamin A and antioxidant from 110, and any excipients can be mixed in emulsion vat 112. All three of these feeds, from line 140, container 110 and mixer 114 can be free of organic solvents. They can be added in any order to emulsion vat 112 and can be added continuously or on a batch basis. The emulsion vat can be pre-charged with an aqueous solution of one, two, three or more polysaccharides, such as starch, maltodextrin, or both. In one set of embodiments, the procedure is carried out on a batch basis where vitamin A palmitate (VAP) and antioxidant is transferred to emulsion vat 112 and emulsified prior to the addition of polymer suspension. The suspension of acidified polymer can be added to the VAP emulsion where the colloidal polymer suspension may self-assemble around the VAP droplets. Emulsion vat 112 includes a mixer capable of emulsifying the vitamin A and the suspended polymer. The mixing can occur, for example, over a temperature range of 10-60° C., 20-50° C., 30-40° C., or 30-35° C. The mixer can be, for example, a blender, a vortex mixer, a rotary mixer or an in-line mixer. In one set of embodiments, the mixer is a high shear dispersion blade mixer. The mixture in emulsion vat 112 can be passed through high-pressure homogenizer 116 to further emulsify the components. In one embodiment, the high-pressure emulsifier is a BOS model MG2-3505. As shown, the mixture can pass through high-pressure homogenizer 116 and then be recycled back to emulsion vat 112. The stability of an emulsion or suspension is dependent on a number of factors including the size of the vitamin A droplets. Droplet size is measured by volume using laser diffraction with the MASTERSIZER® 3000 in standard analysis mode. In general, the smaller a droplet size, the more stable the emulsion. In various embodiments, the median volume D50 droplet size can be less than 1.0 μm, less than 800 nm, less than 600 nm, less than 500 nm, less than 400 nm or less than 350 nm. Size consistency has been shown to be important and a unimodal size distribution around the mean size is believed to produce a better performing product. The standard deviation of vitamin A droplet size can be less than 200 nm, less than 100 nm or less than 50 nm. Emulsion vat 112 can be monitored to evaluate the quality of the emulsion by measuring various parameters including, for example, droplet size, color, pH, temperature and turbidity. When the emulsion has achieved a desired droplet size, e.g., less than 5 μm, less than 1 μm, less than 500 nm, less than 400 nm or less than 350 nm, it can be transferred to drying process 102. In some embodiments, the batch is emulsified for more than 30 min, more than 1 hr, or more than 6 hr.

In other embodiments, a single pass in-line homogenization process can be employed. In this case, an initial emulsion can be generated using an overhead high shear dispersion mixer in a primary tank. This high shear dispersion mixture can obtain a volume median droplet size, for example, less than 50 μm, less than 30 μm or less than 10 μm. To reduce this droplet size, this emulsion is subsequently fed to an inline high shear pump and homogenizer. This reduces the VAP droplet size to, for example, less than 5 μm, less than 1 μm, less than 500 nm, less than 400 nm or less than 350 nm. The emulsion can then be transferred to a spray dryer (or other dryer) for composite particle formation.

In Drying Process 102, the suspension from emulsion vat 112 is flowed to a dryer such as spray dryer 120. Spray dryer 120 includes air input 170 that can supply a heated stream of air. Spray dryer 120 atomizes the liquid that is fed through one or more spraying devices or nozzles 118 and solid particles are formed as water is removed. Spraying devices can be selected from a variety of types, for instance, pressure, air-atomizing, spinning or rotating disk, ultrasonic, piezoelectric and electrostatic. As shown, spray dryer 120 is fed by air stream 170 and includes internal fluid bed dryer 124 with additional fluidizing air stream 172. In some embodiments, the dryer can include no internal bed or can have an external bed 128, fed by air stream 182, or can have both an internal and external bed. Fines that are formed during the drying process are carried via extractors on the roof of the dryer to cyclone separator 126 via conduit 174 and/or from the roof of the external fluid bed dryer 128 via conduit 184. Cyclone separator 126 also includes an exhaust for moisture and air. The captured fines are recycled to spray dryer 120 via conduit 176. These fines are directed to either conduit 178 or 180 depending on the degree of agglomeration desired. In some embodiments, better yield and particle size are obtained when fines are directed through conduit 178 to the upper half or upper third of spray dryer 120. This is believed to be due to larger particle formation and a reduction in caking on the sides of the dryer. The fines can coat the partially dried droplets and lead to less adherence to the dryer walls. Fluid bed dryer 128 separates the desired product, dried vitamin A powder, from dryer fines via gravity. Optionally, a cyclone 130 and/or a bag house filter can be used to collect the product. The collected particles are passed through sieve 132 that can remove particles deemed too large, such as those that are 2×, 5×, or 10×, 50× or 100× the volume median particle size. After sieving, particles are dry and can be stored or incorporated into a foodstuff. Optional postproduction processes can include packaging, milling, sizing, and the addition of components such as colorants, antioxidants, dehydrants, lubricants and other micronutrients.

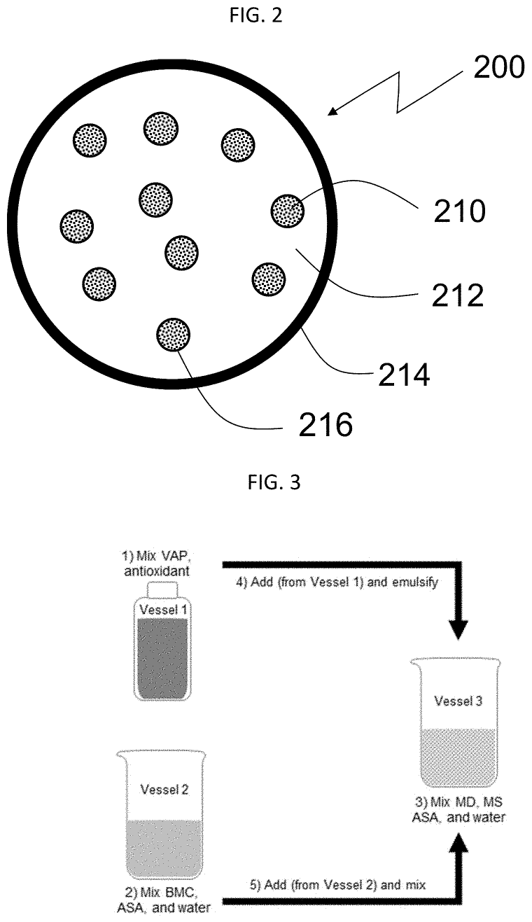

During the production process droplets of vitamin A palmitate (VAP) and suspended polymer droplets comingle to form composite microdroplets of vitamin A and pH sensitive polymer. It is believed that the pH sensitive polymer surrounds the vitamin A droplets as the hydrophobic portions of the polymer are attracted to the fat-soluble vitamin. Once they are emulsified together, the polymer and vitamin A palmitate can form emulsified co-droplets of vitamin A palmitate and associated polymer that can have a volume median diameter of between 50 nm and 2 μm, between 100 nm and 1 μm, between 100 nm and 500 nm, or between 200 nm and 400 nm. In some cases, the resulting dried particle can comprise a contiguous mass of vitamin A embedded in a pH sensitive polymer and have a volume median diameter (D₅₀) between, for instance, 50 and 2000 μm. However, in other cases, when dried, it is believed that these co-droplets of vitamin A and polymer coalesce into composite droplets such as that illustrated in FIG. 2. Note that the figure is an approximation for purposes of illustration and that actual particles may not be spherical and may not contain any specific number of discrete vitamin A subparticles. Composite particle 200 includes individual sub-micron particles of vitamin A 210 that can have a volume median diameter similar to that of the emulsified droplets of vitamin A that are formed during the liquid phase. In the liquid state, the pH sensitive polymer 216 can preferentially arrange around the subdroplets of vitamin A. Once dried, these sub-micron particles of vitamin A can have a volume median diameter D₅₀ of, for example, between 100 and 500 nm, between 200 and 400 nm or from 300 to 450 nm. A plurality of submicron vitamin A particles can be retained together by the polymer and excipients to form a composite particle 200 comprising vitamin A, polymer, antioxidant and excipient(s) 212. In various embodiments these composite particles can have a volume median diameter D₅₀ of between 1 and 1000 μm, 1 and 100 μm, 1 and 10 μm, 50 and 1000 μm, 50 and 400 μm, 100 and 400 μm or 50 and 300 μm. Composite particles may also be coated with a starch coating 214 that can help prevent the composite particles from clumping and agglomerating.

The vitamin A particles described herein can be incorporated into foodstuffs such as bouillon, rice, pasta, flour, sugar, corn meal and other foods that are typically cooked before eating, and/or milk, milk powder, soy sauce, malt beverages, ready-to-use therapeutic foods and other foods or condiments that are not typically cooked before eating. The composite particles of vitamin A can retain more than 50, more than 60, more than 70, more than 80 or more than 90 percent of their vitamin A activity after cooking at 90° C. for 120 minutes. Previously it has been found that state of the art vitamin A particles may exhibit good short-term stability in heated water but that the same particles exhibit poor shelf life under typical storage conditions. In contrast, the composite particles described herein can maintain more than 70, more than 80 or more than 90 percent of their vitamin A activity over a storage period of 28, 60, 90 or 120 days at 40° C. and 75% relative humidity. Particles subjected to both i) storage for 6 months at 40° C. and 75% relative humidity and ii) cooking for 120 minutes at 90° C. can retain more than 40%, more than 50%, more than 60%, more than 70% or more than 80% of their original potency. The particles also show improved stability when other nutritive additives are included. For example, iron has been shown to hinder the shelf life of vitamin A, but that reduction in shelf life is minimized with the current formulations under storage or cooking conditions. Vitamin A particles can be mixed into foodstuffs at concentrations that provide adequate nutrition to those consuming the foodstuffs. For example, the particles can be mixed into a food such as bouillon (wt/wt) at a vitamin A (RE) concentration of from 0-1300 mg RE/kg of fortified foodstuffs, from 13-510 mg RE/kg, from 19-320 mg RE/kg or from 19-200 mg RE/kg. The amounts in wheat flour can be, for example, from 0-46 mg RE/kg, from 0.46-18 mg RE/kg, from 0.69-11 mg RE/kg or from 0.69-7.0 mg RE/kg. The amounts in sugar can be, for example, from 0-120 mg RE/kg, from 1.2-49 mg RE/kg, from 1.8-31 mg RE/kg or from 1.8-19 mg RE/kg. After the particles are mixed with the foodstuff, the resulting fortified material can be packaged and stored. Packaging can be selected to protect the fortified material from moisture and oxygen. The materials added to the fat-soluble vitamin to make the vitamin particles herein can be limited to materials found on the Codex General Standard for Food Additives.

### EXAMPLES

In a set of experiments, production conditions and components were varied to determine the best combination to produce a stable vitamin A particle. Particles were produced essentially as provided above with alterations provided below.

### Emulsion Process

Before the particles can be dried, the liquid vitamin A is emulsified and mixed with the pH sensitive polymer. Several different vitamin emulsion processes were designed and tested in an effort to reduce production time, reduce production costs and to improve the product. All processes described can be purely aqueous in the absence of solvents, mineral acids and surfactants.

Emulsion process #1 is illustrated in FIG. 3 and uses 3 mixing vessels. The first vessel is charged with vitamin A palmitate (VAP) stabilized by antioxidants such as butylated hydroxyanisole (BHA), and/or butylated hydroxytoluene (BHT) or tocopherol (TOC). The first vessel can be heated to form a liquid of the vitamin A/antioxidant mixture. The vitamin A may be provided with an antioxidant, however, the same or a different antioxidant can be added prior to or during the heating process. The second vessel contains i) the pH sensitive polymer, in this case basic methacrylate copolymer (BMC), ii) one or more organic acids, in this case ascorbic acid (ASA), and water. This second vessel is mixed for at least 3.5 hours or at least 12 hours to form a colloidal suspension. The third vessel is first used to mix additional emulsion components such as one or more polysaccharides (maltodextrin (MD), modified starch (MS), an organic acid, e.g., ASA, and water. After completing initial liquid preparation, the stabilized VAP from the first vessel is combined and homogenized using a high-pressure homogenizer (HPH) with the ingredients in vessel three to create an initial VAP emulsion. The BMC colloidal suspension in vessel 2 is then mixed into this VAP emulsion (without HPH) to form the final emulsion to be spray dried. The preparation of the first and second vessels can be completed prior to the combining of all the ingredients. For instance, the first and second vessels can be prepared on a first day, mixed overnight, and then combined on a second day. This method is referred to as the “3-Vessel” protocol.

Emulsion process #2 also uses the 3-Vessel protocol and is the same as process #1 but for the following:

The second vessel (BMC) is mixed for a shorter time period, for instance, less than 3.5 hr, less than 3 hr, less than 2 hr or less than 1 hr. This allows the polymer emulsion to be prepared the same day as the final emulsion, however it does not provide for overnight mixing.

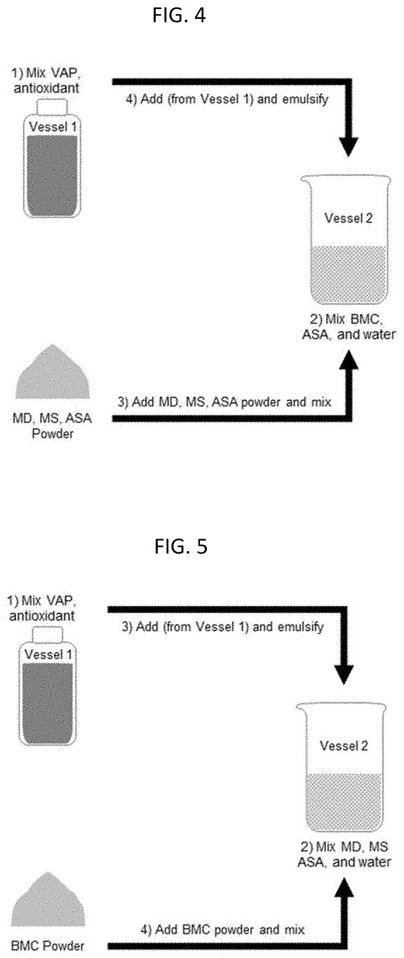

Emulsion process #3 uses a 2-Vessel Protocol shown in FIG. 4 and is carried out as follows:

The first vessel is charged with vitamin A palmitate (VAP) stabilized by antioxidants such as butylated hydroxyanisole (BHA), and/or butylated hydroxytoluene (BHT) or tocopherol (TOC). The first vessel can be heated to form a liquid of the vitamin A/antioxidant mixture. The vitamin A may be provided with an antioxidant, however, the same or a different antioxidant can be added prior to or during the heating process. A second vessel contains i) the pH sensitive polymer, in this case basic methacrylate copolymer (BMC), ii) one or more organic acids, in this case ascorbic acid (ASA), and water. This second vessel is mixed for 0.5 to 3 hours to form a colloidal suspension. To this second vessel is added the additional emulsion components such as one or more polysaccharides (maltodextrin (MD), modified starch (MS)), an organic acid, e.g., ASA, and water. After completing initial liquid preparation, the stabilized VAP from the first vessel is combined and homogenized in vessel 2 using a high-pressure homogenizer (HPH) in the presence of the BMC, modified starch, and maltodextrin, to create a VAP emulsion ready for drying.

Emulsion process #4 is illustrated in FIG. 5, uses a 2-Vessel Protocol and is carried out as follows:

The first vessel is charged with vitamin A palmitate (VAP) stabilized by antioxidants such as butylated hydroxyanisole (BHA), and/or butylated hydroxytoluene (BHT) or tocopherol (TOC). The first vessel can be heated to form a liquid of the vitamin A/antioxidant mixture. The vitamin A may be provided with an antioxidant, however, the same or a different antioxidant can be added prior to or during the heating process. A second vessel is first used to mix additional emulsion components such as one or more polysaccharides (maltodextrin (MD), modified starch (MS), an organic acid, e.g., ASA, and water. After completing initial liquid preparation, the stabilized VAP from the first vessel is combined and homogenized with the ingredients in vessel two using a high-pressure homogenizer (HPH) to create an initial VAP emulsion. To this initial VAP emulsion is added dry BMC (as received). The resulting suspension in vessel two is mixed for more than 0.5, 1, 3 or 6 hours to provide a VAP/BMC emulsion ready for drying.

### Drying Process

After the emulsion of VAP and polymer is formed, the mixture is dried to produce a powdered product. Several different spray drying processes were tested in an effort to reduce cost, improve the product, and evaluate different production scales. Drying can include coating the particles with an agent to improve flow and prevent clumping. Materials such as starch have been found to be useful as a coating. After drying, the powdered product is collected and can optionally be passed through a 10 mm, 5 mm, 2 mm, or 1 mm sieve to remove large aggregates or foreign matter. The material can then be packaged into bags, cartons, drums or other containers, or it can be incorporated into foodstuffs. One packaging process uses aluminum bags that are heat-sealed under vacuum. These packages have been shown to minimize loss of vitamin activity.

Drying process #1 uses a multistage spray dryer and is carried out as follows:

The emulsion is fed to a spray dryer with a pump or pressure, while the emulsion is slightly agitated. The drying system can be identical to, or similar to, the system shown in FIG. 1 and described above. The emulsion is atomized using a nozzle 118 and dried with a heated stream of air 170, and solid particles are formed as water is evaporated. The spray dryer 120 includes an internal fluid bed dryer 124 and optionally an external fluid bed 128. Fines that are formed during the drying process are carried via extractors 174 on the roof of the dryer to a cyclone separator 126, and the captured fines are recycled via conduit 176 to the spray dryer near the nozzle via conduit 178 or to the internal fluid bed chamber via conduit 180, depending on the degree of agglomeration desired. The external fluid bed dryer 128 separates the desired product, dried vitamin A powder, from fines 184. After drying, the powdered product 186 is collected, either directly or with an optional cyclone 130 or bag house filter and may be passed through a sieve 132. The product 188 can then be packaged 134 or incorporated into foodstuffs.

Drying process #2 uses a recirculating starch cloud on a multistage spray dryer and is carried out as follows:

The emulsion is fed to a spray dryer with a pump or pressure, while the emulsion is slightly agitated. The drying system can be identical to, or similar to, the system shown in FIG. 1 and described above. The emulsion is atomized using a nozzle 118 and dried with a heated stream of air 170, and solid particles are formed as water is evaporated. During drying, native starch 154 is fed via conduit 190 into the drying chamber 120 using a feeder 122 to agglomerate and coat particles. The spray dryer includes an internal fluid bed dryer 124 and optionally an external fluid bed 128. Fines that are formed during the drying process, as well as unused native starch particles, are carried via extractors 174 on the roof of the dryer to a cyclone separator 126, and the captured fines are recycled via conduit 176 to the spray dryer near the nozzle via conduit 178 or in the internal fluid bed chamber via conduit 180, depending on the degree of agglomeration desired. The fluid bed dryer 128 separates the desired product, dried vitamin A powder, from fines. After drying, the powdered product 186 is collected, either directly or with an optional cyclone 130 or bag house filter and may be passed through a sieve 132. The product 188 can then be packaged 134 or incorporated into foodstuffs.

Drying process #3 uses a single stage spray dryer and is carried out as follows:

The emulsion is fed to a spray dryer. The emulsion is atomized using a nozzle and dried with a heated stream of air, and solid particles are formed as water is evaporated. The cyclone separates the desired product, dried vitamin A powder, from the exhaust gas. After drying, the powdered product is collected and may be passed through a sieve. The material can then be packaged or incorporated into foodstuffs.

Drying process #4 uses a fluidized bed dryer and is carried out as follows:

The emulsion is fed to a fluidized bed dryer. The emulsion is atomized using a nozzle and dried with a heated stream of air, and solid particles are formed as water is evaporated. The fluidized bed is charged with native starch, which agglomerates with the particles as they dry. After drying, the powdered product is collected and may be passed through a sieve. The material can then be packaged or incorporated into foodstuffs.

After drying, composite particles can have moisture contents of less than 10, less than 5 or less than 3% by weight.

### Particle Forming Examples

### Example 1

104.0 g of BMC were added to a solution of 395.0 g of ultrapure water and 20.5 g of L-ascorbic acid (pH 7.2), then mixed at 20° C. overnight. The following day in a separate vessel, 10.4 g of BHA were added to 104.0 g of BHT-stabilized VAP oil pre-heated to 50° C. The VAP mixture was added to a 50° C. solution containing 14.8 g of Maltodextrin DE19, 133.3 g of HI-CAP 100 modified starch, and 20.5 g of L-ascorbic acid in 197.6 g of ultrapure water and emulsified using an APV Model 2000 high-pressure homogenizer. The BMC dispersion (pH 7.2) was added to this vessel and stirred by an IKA Ultra-Turrax T25 high-shear mixer. The emulsion (pH 6.3) was dried on a Diosna Minilab RC fluidized bed dryer charged with 100.0 g of native starch powder at an inlet air temperature of 75° C. for 43 minutes. A Malvern Mastersizer 3000 was used to determine the volume median droplet diameter and span of the final emulsion (0.4 μm and 1.5, respectively) and the volume median particle diameter and span of the product (502 μm and 1.2, respectively). The residual moisture content was 4.5%.

### Example 2

104.0 g of BMC were added to a solution of 395.1 g of ultrapure water and 20.5 g of L-ascorbic acid, then mixed at 20° C. overnight. The following day in a separate vessel, 10.4 g of BHT were added to 104.0 g of BHT-stabilized VAP oil pre-heated to 50° C. The VAP mixture was added to a 50° C. solution containing 14.8 g of Maltodextrin DE19, 133.3 g of HI-CAP 100 modified starch, and 20.5 g of L-ascorbic acid in 197.5 g of ultrapure water and emulsified using an APV Model 2000 high-pressure homogenizer. The BMC dispersion was added to this vessel and stirred by an IKA Ultra-Turrax T25 high-shear mixer. The emulsion was dried on a Buchi Mini Spray Dryer B-290 at an inlet air temperature of 100° C. and an outlet temperature of 63° C. for 29 minutes. A Malvern Mastersizer 3000 was used to determine the volume median droplet diameter and span of the final emulsion (0.5 μm and 1.9, respectively) and the volume median particle diameter of the product (10 μm). The residual moisture content was 3.2%.

### Example 3

15.1 kg of unstabilized VAP oil were heated in a 60° C. water bath for at least 12 hours, and 1.5 kg of BHT were added. 15.1 kg of BMC were added to 203.0 kg of reverse osmosis (RO) water and 3.0 kg of L-ascorbic acid and stirred at 40° C. for 2.5 hours. 29.6 kg of Maltodextrin DE19, 32.7 kg of HI-CAP 100 modified starch, and 3.0 kg of L-ascorbic acid were added to this vessel, followed by the VAP mixture. Emulsification was performed using a BOS MG2-3505 high-pressure homogenizer. The emulsion was dried on an Entropie Serit SME 180 AB1 pilot multistage spray dryer with an internal fluid bed for 150 minutes. The inlet/outlet air temperature of the spray dry tower were 140° C./68° C., respectively, and the inlet air temperature of the internal fluid bed dryer was 55° C. Fines were recirculated to the internal fluid bed. A Malvern Mastersizer 3000 was used to determine the volume median droplet diameter and span of the final emulsion (0.4 μm and 1.6, respectively) and the volume median particle diameter and span of the product (95 μm and 1.4, respectively). The residual moisture content was 2.2%.

### Example 4

20.4 kg of unstabilized VAP oil were heated with a heating belt set at 70° C. for at least 12 hours, and 2.0 kg of BHT were added. The VAP mixture was added to a 50° C. solution containing 2.9 kg of Maltodextrin DE19, 26.2 kg of HI-CAP 100 modified starch, and 4.0 kg of L-ascorbic acid in 140.0 kg of RO water and emulsified using a BOS MG2-3505 high-pressure homogenizer. 20.4 kg of BMC were added to 60.0 kg of RO water and 4.0 kg of L-ascorbic acid, then mixed at ambient temperature for 3 hours. The BMC dispersion was added to the vessel with the VAP mixture and stirred. The emulsion was dried on an Entropie Serit SME 180 AB1 pilot multistage spray dryer with a recirculating starch cloud and an internal fluid bed. During the 300-minute drying process, 68.9 kg of native starch were added to the drying tower. The inlet/outlet air temperature of the tower were 230° C./90° C., respectively, and the inlet air temperature of the internal fluid bed dryer was 90° C. Fines were recirculated to the internal fluid bed. A Malvern Mastersizer 3000 was used to determine the volume median droplet diameter and span of the final emulsion (0.4 μm and 1.2, respectively) and the volume median particle diameter and span of the product (297 μm and 1.5, respectively). The residual moisture content was 4.2%.

### Example 5

11.3 kg of TOC-stabilized VAP oil were heated with a heating belt set at 70° C. for at least 12 hours, then added to a separate solution containing 33.4 kg of Maltodextrin DE19, 14.5 kg of CAPSUL TA modified starch, and 2.2 kg of L-ascorbic acid in 138.0 kg of RO water and emulsified using a BOS MG2-350S high-pressure homogenizer. 11.3 kg of BMC were added to 45.0 kg of RO water and 2.3 kg of L-ascorbic acid in a separate vessel, then stirred at ambient temperature for 3 hours. The BMC dispersion was added to the VAP mixture and stirred. The emulsion was dried on an Entropie Serit SME 180 AB1 pilot multistage spray dryer with an internal fluid bed for 465 minutes. The inlet/outlet air temperature of the spray dry tower was 155° C./68° C. respectively, and the inlet air temperature of the internal fluid bed dryer was 60° C. Fines were recirculated to the internal fluid bed. A Malvern Mastersizer 3000 was used to determine the volume median droplet diameter and span of the final emulsion (0.4 μm and 1.6, respectively) and the volume median particle diameter and span of the product (86 μm and 1.5, respectively). The residual moisture content was 2.7%.

### Example 6

11.4 kg of unstabilized VAP oil were heated with a heating belt set at 70° C. for at least 12 hours, and 0.7 kg of BHT was added. 11.3 kg of BMC were added to 44.0 kg of RO water and 1.8 kg of L-tartaric acid and stirred at ambient temperature for 3 hours (final pH 6.8). The VAP mixture was added to a 50° C. solution containing 35.3 kg of Maltodextrin DE19 and 14.5 kg of CAPSUL TA modified starch in 140.0 kg of RO water and emulsified using a BOS MG2-350S high-pressure homogenizer. The BMC dispersion was added to this vessel and stirred (final pH 6.17). The emulsion was dried on an Entropie Serit SME 180 AB1 pilot multistage spray dryer with an internal fluid bed for 465 minutes. The inlet/outlet air temperature of the spray dry tower were 146° C./63° C., respectively, and the inlet air temperature of the internal fluid bed dryer was 60° C. Fines were recirculated to the internal fluid bed. A Malvern Mastersizer 3000 was used to determine the volume median droplet diameter and span of the final emulsion (0.4 μm and 1.5, respectively) and the volume median particle diameter and span of the product (71 μm and 1.5, respectively). The residual moisture content was 3.1%.

### Example 7

17.5 kg of unstabilized VAP oil were heated with a heating belt set at 70° C. for at least 12 hours, and 1.0 kg of BHA and 1.0 kg of BHT were added. 17.5 kg of BMC were added to 60.0 kg of RO water and 3.5 kg of L-ascorbic acid and stirred at ambient temperature for 3 hours (final pH 7.08). The VAP mixture was added to a 50° C. solution containing 33.7 kg of Maltodextrin DE19, 22.5 kg of CAPSUL TA modified starch, and 3.5 kg of L-ascorbic acid in 143.0 kg of RO water and emulsified using a BOS MG2-350S high-pressure homogenizer. The BMC dispersion was added to this vessel and stirred (final pH 5.95). The emulsion was dried on an Entropie Serit SME 180 AB1 pilot multistage spray dryer with an internal fluid bed for 585 minutes. The inlet/outlet air temperature of the spray dry tower were 145° C./65° C., respectively, and the inlet air temperature of the internal fluid bed dryer was 60° C. Fines were recirculated to the internal fluid bed. A Malvern Mastersizer 3000 was used to determine the volume median droplet diameter and span of the final emulsion (0.4 μm and 1.9, respectively) and the volume median particle diameter and span of the product (102 μm and 1.6, respectively). The residual moisture content was 2.3%.

### Example 8

16.2 kg of unstabilized VAP oil were heated with a heating belt set at 70° C. for at least 12 hours, and 0.5 kg of BHA and 0.5 kg of BHT were added. The VAP mixture was added to a 50° C. solution containing 16.2 kg of CAPSUL TA modified starch and 3.2 kg of L-ascorbic acid in 140.0 kg of RO water and emulsified using a BOS MG2-350S high-pressure homogenizer. 3.2 kg of L-ascorbic acid and 16.2 kg of BMC powder were added and stirred. The emulsion was dried on an Entropie Serit SME 180 AB1 pilot multistage spray dryer with recirculating starch cloud and an internal fluid bed for 300 minutes. During the drying process, 36.6 kg of native starch were added to the drying tower. The inlet/outlet air temperature of the tower were 220° C./97° C., respectively, and the inlet air temperature of the internal fluid bed dryer was 90° C. Fines were recirculated to the internal fluid bed. A Malvern Mastersizer 3000 was used to determine the volume median droplet diameter of the final emulsion and span (0.4 μm and 2.3, respectively) and the volume median particle diameter and span of the product (262 μm and 1.1, respectively). The residual moisture content was 3.1%.

### Comparative Example 9

18.5 g of 5 M sulfuric acid were added to 413.3 g of ultrapure water containing 5.3 g of Sodium Dodecyl Sulfate and 104.0 g of BMC and mixed at 20° C. overnight. The following day, 14.8 g of Maltodextrin DE19 and 133.3 g of HI-CAP 100 modified starch were added to 206.7 g of ultrapure water, heated to 50° C., and stirred for 30 minutes. 104.0 g of BHT-stabilized VAP oil were added, and the mixture was emulsified using an IKA Ultra-Turrax T25 high-shear mixer for 10 minutes. This mixture was added to the vessel containing the BMC dispersion and emulsified using an IKA Ultra-Turrax T25 high-shear mixer for 5 minutes (final pH 6.8). The emulsion was dried on a Diosna Minilab RC fluidized bed dryer charged with 100.0 g of native starch powder and with an inlet air temperature of 70° C. for 77 minutes. A Malvern Mastersizer 3000 was used to determine the volume median droplet diameter and span of the final emulsion (0.6 μm and 1.3, respectively). The residual moisture content was 2.5%.

### Comparative Example 10

49.0 g of 6 M sulfuric acid were added to 393.7 g of ultrapure water, followed by 5.4 g of Sodium Dodecyl Sulfate and 104.0 g of BMC, and mixed at 20° C. overnight. The following day, 14.8 g of Maltodextrin DE19 and 133.2 g of HI-CAP 100 modified starch were added to 197.0 g of ultrapure water, heated to 50° C., and stirred for 30 minutes. 104.0 g of BHT-stabilized VAP oil were added, and the mixture was emulsified using an IKA Ultra-Turrax T25 high-shear mixer for 5 minutes. This emulsion was added to the BMC dispersion and stirred (final pH 2.0). The emulsion was dried on a Diosna Minilab RC fluidized bed dryer charged with 150.0 g of native starch powder and with an inlet air temperature of 65° C. for 135 minutes. A Malvern Mastersizer 3000 was used to determine the volume median droplet diameter of the final emulsion (0.96 μm). The residual moisture content was 2.5%.

### Example 11

15.2 g of BHA/BHT-stabilized VAP oil was added to a 50° C. solution containing 27.8 g of Maltodextrin DE19, 18.5 g of PE-100 modified starch, and 2.9 g of L-ascorbic acid in 56.7 g of ultrapure water and emulsified using an IKA Eurostar 20 digital overhead stirrer followed by an IKA Ultra-Turrax T25 high-shear mixer. 2.9 g of L-ascorbic acid and 14.4 g of BMC powder were added to this vessel and stirred (final pH 5.5). The emulsion was dried on a Buchi Mini Spray Dryer B-290 with an inlet air temperature of 100° C. and an outlet temperature of 62° C. for 23 minutes. A Malvern Mastersizer 3000 was used to determine the volume median droplet diameter and span of the final emulsion (0.9 μm and 3.9, respectively). The residual moisture content was 4.9%.

### Example 12

202.2 g of BMC were added to 970.0 g of ultrapure water with 40.2 g of L-ascorbic acid and mixed at 40° C. for 3.5 hours. This dispersion (pH 6.2) was stored unagitated and protected from light at 2-8° C. for 10 weeks (pH 6.6 after storage). Separately, 32.1 g of BHA/BHT-stabilized VAP oil was added to a 50° C. solution containing 55.5 g of Maltodextrin DE19, 37.0 g of PE-100 modified starch, and 5.7 g of L-ascorbic acid in 197.0 g of ultrapure water and emulsified using an APV Model 2000 high-pressure homogenizer. 173.0 g of the stored BMC dispersion was added to this vessel and stirred with an IKA Ultra-Turrax T25 high-shear mixer (pH 5.17). The emulsion was dried on a Buchi Mini Spray Dryer B-290 with an inlet air temperature of 100° C. and an outlet temperature of 62° C. for 22 minutes. A Malvern Mastersizer 3000 was used to determine the volume median droplet size and span of the emulsion (0.4 μm and 1.2, respectively). The residual moisture content was 4.3%.

### Comparative Example 13

68.4 g of BMC and 7.6 g of unstabilized VAP oil were added to 1062 g of dichloromethane to form a solution. This feed was atomized using a spinning disk suspended 30′ in the air. An excess amount of native starch was dispersed on a receiving bed below. The partially dried BMC/VAP particles landed on the bed and were coated with starch. The coated particles were collected and sieved to remove excess starch. A Malvern Mastersizer 3000 was used to determine the volume median particle diameter and span of the product (125 μm and 1.4, respectively).

### Example 14

17.5 kg of unstabilized VAP oil were heated with a heating belt set at 60° C. for at least 12 hours, and 1.0 kg of BHA and 1.0 kg of BHT were added. 17.5 kg of BMC were added to 57.0 kg of RO water and 3.5 kg of L-ascorbic acid and stirred at ambient temperature for 3 hours (final pH 7.05). The VAP mixture was added to a 50° C. solution containing 33.7 kg of Maltodextrin DE19, 22.5 kg of PE 100 modified starch, and 3.5 kg of L-ascorbic acid in 137.0 kg of RO water and emulsified using a BOS MG2-350S high-pressure homogenizer. The BMC dispersion was added to this vessel and stirred (final pH 5.9). The emulsion was dried on an Entropie Serit SME 180 AB1 pilot multistage spray dryer with an internal fluid bed for 240 minutes. The inlet/outlet air temperature of the spray dry tower were 160° C./71-78° C., respectively, and the inlet air temperature of the internal fluid bed dryer was 60° C. Fines were recirculated near the nozzle in the spray dry tower. A Malvern Mastersizer 3000 was used to determine the volume median droplet diameter and span of the final emulsion (0.3 μm and 1.3, respectively) and the volume median particle diameter and span of the product (178 μm and 1.2, respectively). The residual moisture content was 3.5%.

### VAP Content Testing

The actual amount of VAP in the composite particles was measured to compare it to the theoretical amount based on what was added to the process. A powder sample was added to a solution comprising 1:9 water:THF with 0.1% BHT, vortexed, and centrifuged at 8,000 RCF for 5 minutes. The supernatant was diluted 10× in ACN with 0.1% BHT, vortexed, filtered through a 0.2 μm filter, and analyzed by HPLC. HPLC is used for all vitamin A measurements herein. Samples were run with a 97:3 methanol:water mobile phase through a C18 reverse phase column and analyzed with an ultraviolet-visible detector at 325 nm. VAP content was calculated against a linear calibration curve. Results are provided in Table 1 and show that all samples contained VAP concentrations that were in the expected range.

TABLE 1

| | VAP Content (% Theoretical ± |
|---|---|
| Example | Standard Deviation) |
| 1 | 104.9 ± 0.4 |
| 2 | 98.3 ± 1.0 |
| 3 | 89.5 ± 0.4 |
| 4 | 84.4 ± 0.4 |
| 5 | 90.5 ± 1.0 |
| 6 | 85.0 ± 0.4 |
| 7 | 106.0 ± 0.4 |
| 8 | 94.0 ± 0.7 |
| 9 | 118.4¹ |
| 10 | 101.3¹ |
| 11 | 94.1 ± 1.2 |
| 12 | 89.5 ± 0.7 |
| 13 | 79.7^{1,2} |
| 14 | 93.3 ± 0.3 |

¹Single measurement only.
²Theoretical content is imprecise due to unknown amount of starch being used to coat particles during spray tower process.

### Cooking Stability Testing

The composite particles of examples 1-14, as well as commercial comparator VAP powders available from BASF, were tested for resistance to degradation under cooking conditions. 20 mg of powder were added to 2.0 mL of water and vortexed. This suspension was heated at 90° C. for 2 hours while mixing at 500 rpm using an Eppendorf ThermoMixer C. The mixture was cooled to room temperature, frozen, and lyophilized for at least 24 hours. The resulting powder was analyzed for vitamin A content as performed in above and reported in Table 2 as the percent of the amount measured prior to cooking. The data show that the composite particles (except for comparison examples 10 and 13) lost much less active vitamin A than did the powder. While the commercial comparator powders lost from 30 to 60% of the initial vitamin A, the test examples showed from 0 to 16% loss, or less than about half the amount lost by the commercial comparator powders.

TABLE 2

| Example | VAP Content (% of Initial) |
|---|---|
| 1 | 98.5 |
| 2 | 96.1 |
| 3 | 84.0 |
| 4 | 86.1 |
| 5 | 99.8 |
| 6 | 94.3 |
| 7 | 96.7 |
| 8 | 93.0 |
| 9 | 100.6 |
| 10 | 15.3 |
| 11 | 89.9 |
| 12 | 97.7 |
| 13 | 76.4 ± 8.1¹ |
| 14 | 98.6 |
| BASF-VAP250-CWD MS | 70.0 |
| BASF-VAP250-FG | 41.0 |

¹Measured with multiple replicates and reported as the mean ± the standard deviation. All other samples were single measurement only.

### Storage Stability Testing

To test storage stability when exposed to the environment, 10 samples were exposed to 40° C. for a time period of 28 to 36 days. One g each of samples 1-10, 13 and 14 was evenly distributed in a 4.5 cm diameter open petri dish and stored at 40° C. and ambient relative humidity in a Memmert UF75 universal oven for at least 4 weeks. The powder was analyzed for vitamin A content as performed above. Table 3 illustrates results that show retention between 44 and 91% of the initial vitamin A concentrations.

TABLE 3

| | VAP Content (% Initial ± | Storage Time |
|---|---|---|
| Example | Standard Deviation) | (Days) |
| 1 | 80.3 ± 0.6 | 35 |
| 2 | 44.3 ± 0.6 | 36 |
| 3 | 91.0 ± 1.7 | 31 |
| 4 | 88.7 ± 0.6 | 34 |
| 5 | 65.0 ± 1.0 | 29 |
| 6 | 89.3 ± 0.6 | 28 |
| 7 | 88.7 ± 1.5 | 28 |
| 8 | 80.3 ± 0.6 | 35 |
| 9 | 58.3 ± 0.6 | 30 |
| 10 | 80.0 ± 0.6 | 30 |
| 13 | 48.4 ± 0.4 | 30 |
| 14 | 91.0 ± 0.0 | 30 |

### Shelf Stability

Six of the examples were tested for long term storage when stored in packaging appropriate for vitamin A and for foodstuffs containing vitamin A. 20-30 g samples of powders were placed into vacuum-sealed aluminum bags and stored at ambient conditions for 6 months. The powder was analyzed for vitamin A content as performed above. The results provided in Table 4 indicate retention of at least 95% for the experimental samples tested.

TABLE 4

| | VAP Content after 6 Months of Storage |
|---|---|
| Example | (% Initial ± Standard Deviation) |
| 3 | 97.3 ± 0.6 |
| 4 | 95.3 ± 0.6 |
| 5 | 100.7 ± 0.6 |
| 6 | 103.3 ± 0.6 |
| 7 | 97.7 ± 0.6 |
| 8 | 101.0 ± 1.7 |

### Bouillon Stability Study 1

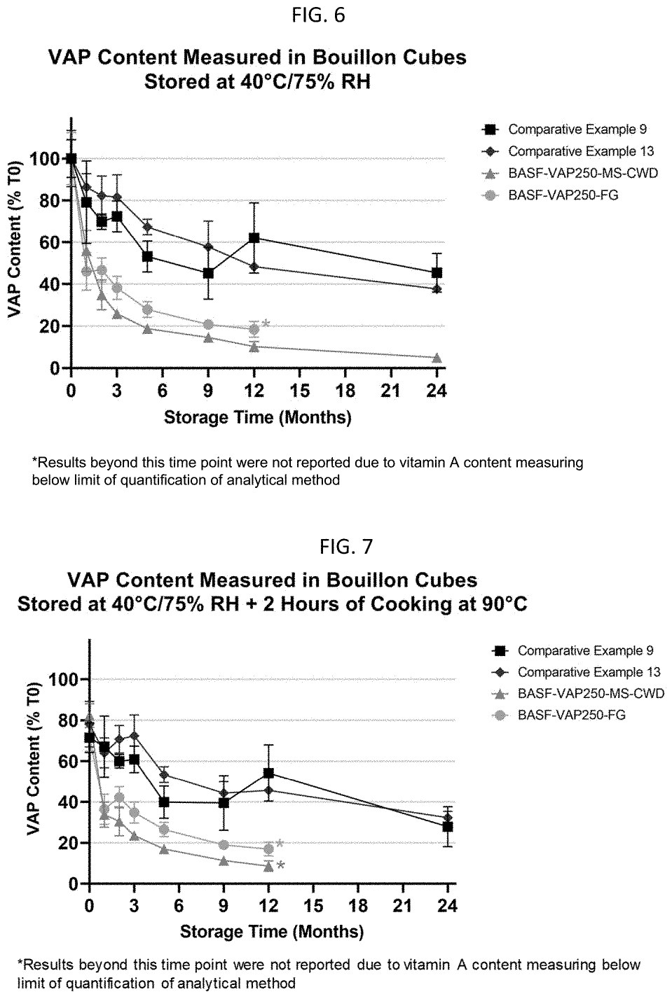

VAP composite particles from Comparative Examples 9 and 13, as well as commercial comparators (BASF-VAP250-FG and BASF-VAP250-MS-CWD), were combined with Nestlé Maggi Star bouillon powder (0.67 mg VAP per g bouillon) and mixed until uniformly dispersed. 4 g fortified bouillon tablets were formed using a punch and die set on a manual tablet press. The tablets were stored at 40° C./75% RH, in accordance with the ICH guideline for accelerated stability testing for all world zones. Tablets were sampled periodically for VAP content and cooking stability analyses. Content analysis was performed by dissolving bouillon tablets in water at a concentration of 91 mg/mL, freezing a 4-10 mL sample, lyophilizing for at least 24 hours, and then analyzing the powder for vitamin A content as described above. Cooking stability analysis was performed by dissolving bouillon tablets in water at a concentration of 91 mg/mL, diluting 4-5× in water, and heating to 90° C. for 2 hours. The total sample mass was maintained over the cooking duration. A 10 mL sample was frozen and lyophilized for at least 72 hours. The powder was analyzed for vitamin A content as described above. The 24-month stability results are shown in FIG. 6, with error bars representing standard deviation. The results indicate retention of 40% or more for the comparative examples while the commercial comparator powders were degraded to a level of less than 10% of its original vitamin A activity. The 24-month stability results after storage plus 2 hours of cooking at 90° C. are shown in FIG. 7, with error bars representing standard deviation. The results indicate retention of 30% or more for the comparative examples while the commercial comparator powders were degraded below the level of quantification of the analytical method.

### Bouillon Stability Study 2

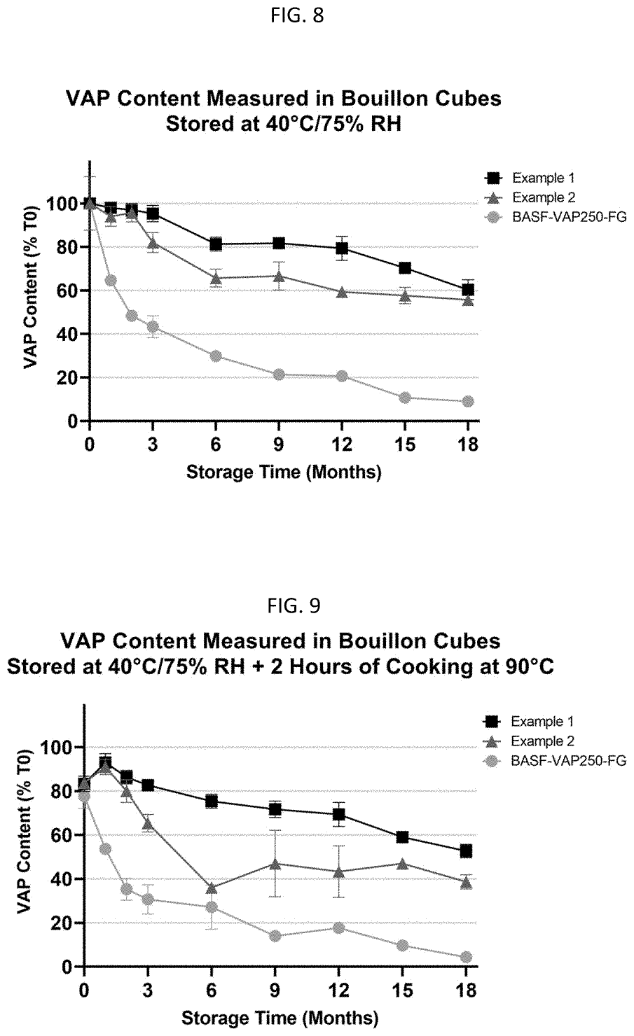

VAP composite particles from Examples 1 and 2, as well as a commercial comparator (BASF-VAP250-FG), were combined with Nestlé Maggi Star bouillon powder (0.67 mg VAP per g bouillon) and mixed until uniformly dispersed. 4 g fortified bouillon tablets were formed using a punch and die set on a manual tablet press. The tablets were stored at 40° C./75% RH, in accordance with the ICH guideline for accelerated stability testing for all world zones. Tablets were sampled periodically for VAP content and cooking stability analyses per the testing procedures described in Bouillon Stability Study 1. The 18-month stability results are shown in FIG. 8, with error bars representing standard deviation. The results indicate retention of 60% or more for the experimental examples while the commercial comparator powder was degraded to a level of about 10% of its original vitamin A activity. The 18-month stability results after storage plus 2 hours of cooking at 90° C. are shown in FIG. 9, with error bars representing standard deviation. The results indicate retention of 40% or more for Examples 1 and 2 while the commercial comparator powder was degraded to a level less than 10% of its original vitamin A activity.

### Bouillon Stability Study 3

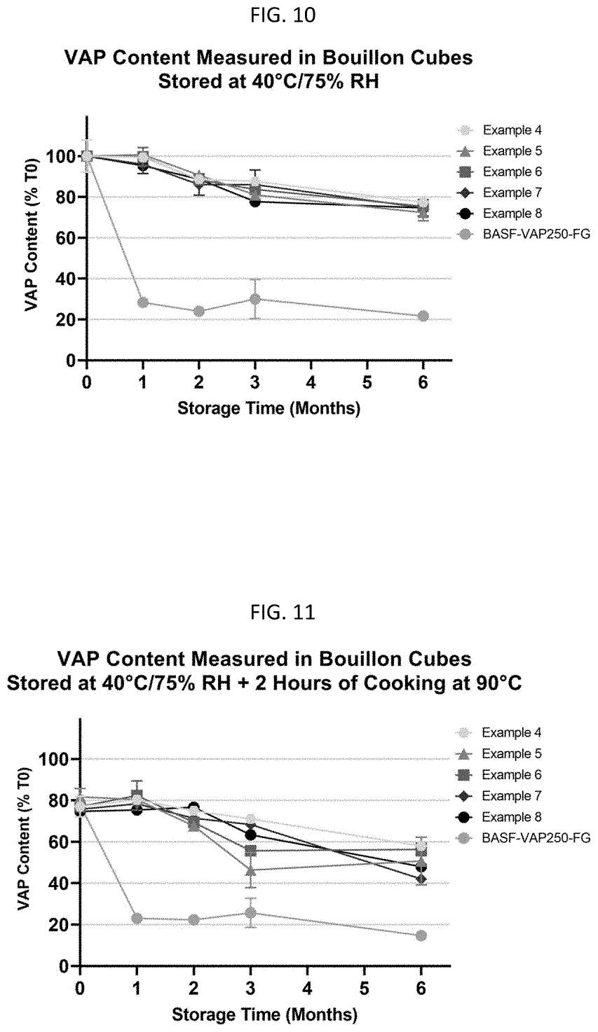

VAP composite particles from Examples 4-8, as well as a commercial comparator (BASF-VAP250-FG), were combined with Nestlé Maggi Star bouillon powder (0.35 mg VAP per g bouillon) and mixed until uniformly dispersed. 11 g fortified bouillon tablets were formed using a Bonals Technologies P40 rotary press and wrapped using a Theegarten BCW3 wrapping machine. The tablets were stored at 40° C./75% RH, in accordance with the ICH guideline for accelerated stability testing for all world zones. Tablets were sampled periodically for VAP content and cooking stability analyses per the testing procedures described in Bouillon Stability Study 1. The 6-month stability results are shown in FIG. 10, with error bars representing standard deviation. The results indicate retention of 70% or more for the experimental examples while the commercial comparator powder was degraded to a level of about 25% of its original vitamin A activity. The 6-month stability results after storage plus 2 hours of cooking at 90° C. are shown in FIG. 11, with error bars representing standard deviation. The results indicate retention of 40% or more for Examples 4-8 while the commercial comparator powder was degraded to a level less than 20% of its original vitamin A activity.

Results from the bouillon stability studies described above are provided in Table 5 and Table 6, below. Table 5 provides the amount of VAP recovered after the storage times provided in the table. Table 6 provides the amount of VAP recovered after cooking the samples for two hours at 90° C., after the storage times provided in the table. Results from Examples 1 and 2 show stability equal to, or better than, that of Comparative Example 9 (mineral acid and SDS surfactant) and Comparative Example 13 (organic solvent). The experimental examples indicate that the described processes provide for stable vitamin A composite particles without requiring the use of mineral acids, surfactants or organic solvents. All materials used to produce these experimental examples are found in the Codex General Standard for Food Additives.

TABLE 5

| Content | Storage Duration at 40° C./75% RH | | | | |
|---|---|---|---|---|---|
| Example | 3 months | 9 months | 12 months | 18 months | 24 months |
| 1 | 95.3 ± 3.8 | 81.7 ± 2.5 | 79.3 ± 5.5 | 60.3 ± 4.6 | N/A |
| 2 | 82 ± 4.6 | 66.7 ± 6.5 | 59.3 ± 1.5 | 55.7 ± 1.5 | N/A |
| 4 | 87.7 ± 1.5 | N/A | N/A | N/A | N/A |
| 5 | 81.0 ± 1.0 | N/A | N/A | N/A | N/A |
| 6 | 83.7 ± 2.1 | N/A | N/A | N/A | N/A |
| 7 | 86.0 ± 7.2 | N/A | N/A | N/A | N/A |
| 8 | 77.7 ± 2.1 | N/A | N/A | N/A | N/A |
| 9 | 72.4 ± 7.3 | 45.2 ± 12.4 | 62.1 ± 16.8 | N/A | 45.4 ± 9.2 |
| 13 | 81.5 ± 10.7 | 57.7 ± 12.4 | 48.3 ± 1.8 | N/A | 37.7 ± 1.1 |
| BASF- | 25.9 ± 1.9 | 14.6 ± 0.4 | 10.2 ± 2.4 | N/A | 4.9 ± 0.2 |
| VAP250- | | | | | |
| CWD | | | | | |
| MS | | | | | |
| BASF- | 38.2 ± 5.5 | 20.7 ± 1.1 | 18.5 ± 3.8 | N/A | N/R* |
| VAP250-FG | | | | | |

*N/R indicates that the results were not reported due to vitamin A content measuring below the limit of quantification of analytical method

TABLE 6

| Cooking | Storage Duration at 40° C./75% RH | | | | |
|---|---|---|---|---|---|
| Example | 3 months | 9 months | 12 months | 18 months | 24 months |
| 1 | 82.7 ± 1.5 | 71.7 ± 3.8 | 69.3 ± 5.5 | 52.7 ± 3.1 | N/A |
| 2 | 65.3 ± 4.0 | 47.0 ± 15.1 | 43.3 ± 11.7 | 38.7 ± 3.2 | N/A |
| 4 | 71.0 ± 1.0 | N/A | N/A | N/A | N/A |
| 5 | 46.3 ± 8.5 | N/A | N/A | N/A | N/A |
| 6 | 55.7 ± 2.1 | N/A | N/A | N/A | N/A |
| 7 | 68.3 ± 5.9 | N/A | N/A | N/A | N/A |
| 8 | 63.3 ± 3.1 | N/A | N/A | N/A | N/A |
| 9 | 60.1 ± 6.5 | 39.5 ± 13.4 | 54.2 ± 13.8 | N/A | 27.9 ± 9.8 |
| 13 | 72.4 ± 10.2 | 44.7 ± 5.6 | 45.7 ± 1.6 | N/A | 32.3 ± 3.1 |
| BASF- | 23.5 ± 2.1 | 11.4 ± 0.2 | 8.6 ± 2.5 | N/A | N/R* |
| VAP250- | | | | | |
| CWD | | | | | |
| MS | | | | | |
| BASF- | 34.8 ± 5.1 | 18.9 ± 1.4 | 16.9 ± 3.4 | N/A | N/R* |
| VAP250-FG | | | | | |

*N/R indicates that the results were not reported due to vitamin A content measuring below the limit of quantification of analytical method

### Subparticle Size Measurement

Powders were dispersed in hot water to determine the subparticle size. 1 g of powder was added to 20 g of water preheated to 50° C. and stirred. A Malvern Mastersizer 3000 was used to determine the volume median subparticle size and span. The powder from example 12 had a volume median subparticle size of 0.4 μm and a span of 1.3, while the powder from example 14 had a volume median subparticle size of 0.4 μm and a span of 1.2.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

All references, patents and patent applications and publications that are cited or referred to in this application are incorporated in their entirety herein by reference.

1. A composite particle of vitamin A, or a derivative thereof, the particle comprising:
a pH sensitive polymer; and
subparticles or subdroplets of vitamin A embedded in the pH sensitive polymer, wherein
the composite particle is essentially free of organic solvents and mineral acids and an amount of vitamin A in the composite particle is a first concentration after production of the particle and a second concentration after exposing the composite particle to both a shelf life test at 40° C. for 4 weeks and a cooking test in water at 90° C. for 2 hours, and the second concentration is more than 60%, more than 70%, more than 80% or more than 90% of the first concentration.

2. The composite particle of claim 1 comprising vitamin A palmitate, ascorbic acid, maltodextrin and modified starch.

3. The composite particle of claim 1 further comprising ascorbic acid, BHT, BHA, tocopherol, or a combination thereof.

4. The composite particle of claim 1 wherein the composite particle is essentially free of surfactants.

5. The composite particle of claim 1 wherein the pH sensitive polymer comprises a polymethacrylate.

6. The composite particle of claim 5 wherein the pH sensitive polymer comprises BMC.

7. The composite particle of claim 1 wherein the subparticles or subdroplets have a median volume diameter D₅₀ of between 50 nm and 2 μm, between 100 nm and 1 μm, between 100 nm and 500 nm, or between 200 nm and 400 nm.

8. The composite particle of claim 1 wherein the composite particle has a median volume diameter, D₅₀, of between 1 and 1000 μm, 1 and 100 μm, 1 and 10 μm, 50 and 1000 μm, 50 and 400 μm, 100 and 400 μm, 50 and 300 μm or 100 and 500 μm.

9. The composite particle of claim 7 wherein the composite particle has a standard deviation of the volume diameter of less than 50 microns.

10. The composite particle of claim 1 wherein the composite particle comprises vitamin C.

11. A bouillon comprising the composite particle of claim 1.

12. Wheat flour, millet flour, cassava flour, tapioca flour, teff flour, corn meal, milk, milk powder, malt beverages, soy sauce, ready-to-use therapeutic foods, rice, or sugar comprising the composite particle of claim 1.

13. A method comprising:
forming an acidified aqueous vehicle at a pH of less than or equal to 6.0;
combining a pH sensitive polymer with the aqueous vehicle to form a colloidal suspension;
allowing the pH of the suspension to rise to greater than 6.0;
emulsifying a fat-soluble vitamin or derivative thereof;
combining the colloidal suspension and the emulsified fat-soluble vitamin or derivative thereof to form a dispersion;
limiting the increase in pH after addition of the fat-soluble vitamin or derivative thereof to a pH of 7.0; and
removing water from the dispersion to produce composite microparticles comprising a matrix of fat-soluble vitamin, or derivatives thereof, and a pH sensitive polymer.

14. The method of claim 13 wherein the water is removed by spray drying, fluid bed drying, vacuum drying, rotary evaporator drying, lyophilizing and/or multistage drying.

15. The method of claim 13 wherein the method is free of at least one of surfactants and mineral acids.

16. The method of claim 13 wherein the pH of the acidified aqueous vehicle is reduced to 5.5 or less using an organic acid.

17. The method of claim 16 wherein the organic acid is ascorbic acid, tartaric acid or both.

18. The method of claim 13 wherein the method is free of organic solvents.

19. The method of claim 13 wherein the fat-soluble vitamin is emulsified in an aqueous solution comprising maltodextrin, starch, or a combination thereof.

20. The method of claim 13 comprising coating the composite microparticles with a polysaccharide.

21-60. (canceled)
